(19) United States
(12) Patent Application Publication
(10) Pub. No.: US 2012/0020947 A1
(43) Pub. Date: Jan. 26, 2012

(54) COMPOSITIONS AND METHODS FOR INCREASING LEAN MUSCLE MASS AFTER EXERCISE

(75) Inventors: Shawn Shirazi, Oakville (CA); Phil Apong, Oakville (CA); James Akrong, Oakville (CA)

(73) Assignee: NORTHERN INNOVATIONS AND FORMULATIONS CORP., Oakville (CA)

(21) Appl. No.: 13/099,421

(22) Filed: May 3, 2011

Related U.S. Application Data
(60) Provisional application No. 61/366,735, filed on Jul. 22, 2010.

(51) Int. Cl. A61K 38/48; A61P 21/06

(52) U.S. Cl. 424/94.63; 424/94.64; 424/94.66; 424/94.65

(57) ABSTRACT

The present invention relates to novel methods and compositions comprising casein, creatine or derivatives of creatine, branched chain amino acids and proteases useful for increasing lean muscle mass after exercise.

### FIELD OF THE INVENTION

The present invention relates to novel methods and compositions comprising casein, creatine or derivatives of creatine, branched chain amino acids and proteases useful for increasing lean muscle mass after exercise.

### BACKGROUND OF THE INVENTION

Feeding and resistance exercise are two of the most potent stimulators of skeletal muscle protein synthesis (MPS). Food supplements for enhancing an athlete's muscle size and strength have become popular substitutes for steroids and other drugs in various sports and body building regimes. However, as athletes continually strive for improved performance, there is a continuing need for supplements for improving lean muscle mass and strength.

One factor which enables athletes to participate effectively is a high degree of development of the aerobic capacity and/or strength of skeletal muscle. Strength is a function of training and of muscle mass which requires a net synthesis of proteins in the muscle. Strenuous exercise is an effective stimulus for protein synthesis. Exercise is the repeated use or activity of a muscle group or organ. Exercise is bodily exertion for the sake of developing and maintaining physical fitness. Anaerobic exercise occurs when the activity results in the body incurring an oxygen debt. In contrast, aerobic exercise is physical conditioning involving exercise that does not cause an oxygen debt, such as distance running, jogging, walking, swimming, circuit training or cross country skiing strenuously performed so as to cause a marked, but steady increase in respiration and heart rate over an extended period of time. The most familiar form of anaerobic exercise is weightlifting; but football and tennis are other examples.

Athletes engage in strenuous training to accomplish the goals of their sport. In the period after strenuous exercise, muscle tissue enters a stage of rapid nitrogen absorption in the form of amino acids and small peptides in order to rebuild the muscle fibres, grow and add new muscle fibers. Athletes sometimes enter into a catabolic state during this period. For example, this can happen during sleep after the release of the body's growth hormone (GH) has abated, and drastically cuts the body's ability to synthesize new protein.

Human growth hormone is secreted in a pulsatile fashion following a circadian rhythm. GH has many varied roles throughout life, including increasing rate of protein synthesis and increased turnover of muscle, bone and collagen to the regulation of metabolic function including increased fat metabolism. It is known that about 90% of the body's daily supply of growth hormone is released during the first four (4) hours that a person sleeps. Thus, during sleep, if proteins and nutrients are made readily available to muscles, the body can successfully perform the muscle-repairing, muscle-rebuilding processes that ultimately result in increased muscle size and strength.

Some protein supplements in the form of solid foods and drinks abound. Therefore, one might naturally attempt to wake up during the night to consume one of the many protein supplements during this four (4) hour peak of growth hormone production. However, this method may not work and may be counterproductive to gaining lean muscle mass because many of these supplements have been designed and developed for rapid absorption.

Specialized “pre-workout” and “post-workout” protein supplements are very effective in enhancing muscle growth when used just before and just after strength training, but these may not be optimum when it comes to mitigating sleep-induced muscle catabolism because the proteins are digested and assimilated so rapidly. In addition, the proteins in typical protein supplements are rapidly used up after only the first hour or so of sleep, leaving little protein to be used during the most productive period of slow-wave sleep, when about 90% of the body's growth hormone is released. Waking up in the middle of the night to consume another serving of a typical protein supplement is ultimately counterproductive, since the practice results in disruption of sleep patterns and increased cortisone from the stress accompanied with sleep loss. These stress factors greatly reduce the probability that the protein serving will contribute to protein synthesis and will also disrupt whatever constructive synthesis may be taking place at the moment of waking. The only viable solution to the problem is invention envisioned herein, whereby proteins and carbohydrates are provided in a manner that doesn't disrupt slow wave sleep, GH release or insulin-like growth factor-1 (IGF-I) metabolism, but works in concert to encourage growth of muscle and promote a feeling of relaxation during periods of rest.

Therefore, in order to maximize muscle growth, necessary proteins and nutrients need to be provided to the body to utilize during this growth hormone spike and subsequent increase in endogenous IGF-I levels that occurs secondary to the growth hormone a spike and lasts for several hours. Providing proteins and nutrients throughout the night time period is important in order to make full use of the anabolic and anticatabolic properties of both GH and IGF-I. In this way, muscle and strength loss due to sleep induced catabolism may be reduced. That is, the body needs to be provided with the proper building blocks during the rest stage in order to maximize muscle growth.

Muscle catabolism occurs when the athlete enters a negative nitrogen balance. In contrast, a positive nitrogen balance means the animal has enough nitrogen left over to synthesize muscle proteins. To grow, muscle requires a large array of nutrients, including amino acids (which are derived from protein) for protein synthesis. These nutrient substrates can be supplied by ingesting diets which provide the necessary amounts of protein (the source of amino acids), calories and other nutrients.

The ingestion of meals consisting of proteins causes an increase in the plasma amino acid level. This rise in the availability of amino acids is associated with a rearrangement of the various components of protein metabolism (protein degradation, protein synthesis, amino acid oxidation). It has been shown that postprandial protein gain depended on the rate of digestion of the ingested proteins (period between ingestion and absorption of the nutrients by the body).

Some proteins with a fast rate of digestion, such as whey proteins, have a high nutritive value, that is to say an adequate and balanced supply of amino acids which are essential for t he human body, such as valine, leucine, isoleucine, phenylalanine, lysine, methionine, tryptophan and threonine. However, in spite of this beneficial amino acid balance, the body's use of the amino acids derived from these proteins may not always be optimum because they may be digested too rapidly for certain times of the day.

There is also a need for a nutritional supplement that increases muscle strength during periods when training efforts have stopped (i.e. during sleep or exercise during periods of rest between physical workouts) and the body in a state of repair and growth. It is during this period of repair and growth that there is a desire to prolong the rate of protein digestion over an extended period because it is important that the muscle cells have available sufficient levels of nitrogen in the form of amino acids.

It is also desirable to apply training programs which employ a combination of specific exercise technique and diet. Many nutrients can be supplemented to athletes in training. The present invention is directed at compositions and methods for supplementing the diet of an athlete for increasing muscle mass, muscle size and strength after exercise and/or between physical workouts.

### SUMMARY OF THE INVENTION

It is an object of the present invention to provide for compositions and methods for increasing lean muscle mass.

According to an aspect of the present invention, there is provided a composition and a method for increasing lean muscle mass during periods of rest after exercise.

According to an aspect of the present invention, there is provided a composition and a method for maintaining lean muscle mass during extended periods of rest from exercise.

According to an aspect of the present invention, there is provided a composition and a method for maintaining lean muscle mass during recovery from exercise-induced injury.

According to an aspect of the present invention, there is provided a composition and a method for increasing lean muscle mass and suppressing exercise-induced nausea.

According to an aspect of the present invention, there is provided a composition and a method for increasing lean muscle mass and suppressing exercise-induced anxiety.

According to an aspect of the present invention, there is provided a composition and a method for increasing lean muscle mass and suppressing exercise-induced excessive inflammation.

According to an aspect of the invention, there is provided a dietary supplement comprising casein, creatine or derivatives thereof, at least one branched chain amino acid and at least one protease.

According to an aspect of the invention, there is provided a comestible composition comprising casein, creatine or derivatives thereof, at least one branched chain amino acid and at least one protease.

According to an aspect of the invention, there is provided a method for increasing muscle mass after physical exercise comprising providing a comestible composition comprising casein, creatine or derivatives thereof, at least one branched chain amino acid and at least one protease.

According to an aspect of the invention, there is provided a method for increasing muscle mass after physical exercise comprising administering a comestible composition comprising casein, creatine or derivatives thereof, at least one branched chain amino acid and at least one protease to an individual in need thereof.

According to an aspect of the invention, there is provided a comestible composition comprising casein, creatine or derivatives thereof, at least one branched chain amino acid and at least one protease for increasing skeletal muscle mass after physical exercise.

According to an aspect of the invention, there is provided a comestible composition comprising casein, creatine or derivatives thereof, at least one branched chain amino acid and at least one protease for use in the preparation of a medicament for increasing muscle mass after physical exercise.

### DETAILED DESCRIPTION OF THE EMBODIMENTS

The form of delivery of dietary protein is known to influence protein digestion, metabolism, and homeostasis at several levels, starting with the gastric emptying and absorption rates of amino acids, which, in turn, influence their postprandial metabolic orientation essentially through effects on kinetics of amino acid uptake and transfer to catabolic and anabolic pathways and subsequent protein retention. The rapidly absorbed amino acids (either from mixtures, from peptides, or released from “fast” proteins) produce lower body protein retention in the postprandial phase in healthy young adults than do slowly absorbed amino acids released from “slow” proteins.

Slow absorption rates of protein in the bloodstream are ideal between meals and often between workouts. The slow digestion and delivery of protein provides a steady supply of amino acids to the skeletal muscles. For example, it has been suggested that milk promotes better whole body nitrogen retention at rest, and greater skeletal muscle protein accretion after resistance exercise, compared with soy protein. The difference in the metabolism of milk and soy proteins has been attributed to their digestion kinetics, wherein milk is digested slower than soy. Milk contains two protein fractions, whey and casein, which are characterized based on their rate of digestion as fast and slow proteins, respectively. Whey protein is acid soluble and thus is digested quickly and results in a pronounced aminoacidemia. Data obtained at the whole body level show that whey (similar to soy) induces a transient rise in whole body protein synthesis and leucine oxidation at rest. Conversely, casein has a modest effect on whole body protein synthesis but instead inhibits whole body protein breakdown.

It is known that after whey ingestion, the plasma appearance of dietary amino acids is fast, high, and transient. This amino acid pattern is associated with an increased protein synthesis and oxidation and no change in protein breakdown. By contrast, the plasma appearance of dietary amino acids after casein ingestion is slower, lower, and prolonged with a different whole body metabolic response: protein synthesis slightly increases, oxidation is moderately stimulated, but protein breakdown is markedly inhibited.

A unique feature of the primary structures of caseins, which are most likely responsible for their functional properties, is the distinct amphipathic nature of their sequences. These structures suggest that their tertiary structures are organized into polar and hydrophobic domains, this structural characteristic being responsible for the unique interactions that define the micelle structure of the caseins. The present invention also includes casein hydrolyzed by various proteases such as, but not limited to, endopeptidases and proline-specific endopeptidases.

According to an embodiment, the present the invention includes salts of casein, micellar casein, hydrolyzed casein or combinations thereof. In embodiments, the calcium salt of casein, calcium caseinate, is used. In embodiments, a combination of micellar and calcium caseinate is used. It has been found that specific ratios of micellar and calcium caseinate are preferred because of improved texture and viscosity. In a preferred embodiment, the ratio of micellar:calcium caseinate is 10-90:90-10. In a preferred embodiment, the ratio of micellar:calcium caseinate is 50:50. In a most preferred embodiment, the ratio of micellar:calcium caseinate is 60:40.

To ensure a steady supply of anabolic proteins that is required after intense physical exercise the use of branched-chain amino acids (BCAAs) is also contemplated. The BCAAs provide a substrate which is utilized instead of muscle mass and liver proteins. BCAAs consist of leucine, isoleucine and valine and are considered essential and despite their importance, humans cannot synthesis them. As such these amino acids must be obtained from the diet. In fact, these amino acids constitute about one-third of skeletal muscle protein and are not only used in the synthesis of other amino acids but they are important in the regulation of the anabolic process in skeletal muscle. It is now known that BCAAs not only increase the rate of protein synthesis but importantly, also inhibit protein degradation.

L-leucine is also known as 2-amino-4 methylvaleric acid, alpha-aminoisocaproic acid and (S)-2-amino-4-methylpentanoic acid. Although the present invention is not to be bound by any theoretical explanation, it is believed that L-leucine is involved in muscle synthesis, as well as helping to promote healing of muscle, bone, and skin tissue. L-leucine is a three branched-chain amino acid which allows it to be used as an energy source by muscle tissue. Leucine plays an important role as nutrient signals which facilitate protein synthesis via mechanisms such as stimulating insulin release which in turn translates to positive influences on muscle growth and inhibition of muscle breakdown; and or directly activating molecules involved in protein synthesis. Insulin production, as set forth in the present invention, in conjunction with the direct signaling effect of amino acids, will work together to directly modify critical control points in muscle to activate the protein kinase mTOR (mammalian target of rapamycin), a site of integration of signals that stimulates muscle protein synthesis.

More specifically, leucine will work via indirect mechanisms to augment protein synthesis via multiple pathways. This anabolic signal, in combination with the known benefits of creatine supplementation, will have an additive affect on changing body composition, e.g., weight loss, and athletic performance, by the addition of lean mass. Administration of leucine has been shown to stimulate protein synthesis in the skeletal muscle of rats. Without being limited to a particular mechanism, leucine activates mTOR which triggers the phosphorylation of 4E-BP1 and S6k1 (and other key protein kinases, i.e. p70S6K), leading to the release of eIF4E (enhancing association of eIF4E with eIF4G) and ultimately leading to increased protein synthesis and inhibition of protein breakdown.

L-valine is also known as 2-aminoisovaleric acid, 2-amino-3-methylbutyric acid, alpha-aminoisovaleric acid and (S)-2-amino-3-methylbutanoic acid. Although the present invention is not to be bound by any theoretical explanation, it is believed that L-valine aids in muscle metabolism, tissue repair, and helps maintain proper nitrogen balance in the body. L-valine is a three branched-chain amino acid which allows it to be used as an energy source by muscle tissue.

L-isoleucine is also known as 2-amino-3-methylvaleric acid, alpha-amino-beta-methylvaleric acid and (2S, 3S)-2-amino-3-methylpentanoic acid. Although the present invention is not to be bound by any theoretical explanation, it is believed that L-isoleucine increases energy levels, enhances endurance, and is necessary for hemoglobin formation.

BCAA ingestion is beneficial during aerobic exercise. When BCAAs are taken during aerobic exercise the net rate of protein degradation has been shown to decrease. The first metabolic reaction in oxidative catabolism of BCAA is transamination (enzymatic transfer of the alpha-amino group to another molecule) resulting in the formation of a branched keto acid and another amino acid. The branched keto acid can either accept an amino group, thus becoming a BCAA again; or be further and irreversibly catabolized for calories. The branched keto acids are also catabolized within muscle cells, albeit to a lesser extent. The major quantity of branched keto acid is exported from muscle via the blood to the liver and kidney where they are catabolized or re-aminated.

Strenuous exercise increases the oxidation of BCAA and it known that trained muscle, while in the resting (non-exercising state), also oxidizes more BCAA than non-trained muscle. Further, it is now known that the BCAA oxidized in skeletal muscle during exercise is derived from muscle protein which is degraded during exercise, as well as from BCAA delivered to the muscle in the bloodstream. Thus, the major source of the blood-borne BCAA during exercise is the liver.

Thus, it is known that exercise causes transient periods wherein the normal balance in skeletal muscle of protein synthesis and degradation has been tipped toward a net, or relative, increase in protein degradation. That is, strenuous exercise causes muscle to burn a portion of its protein structure.

The reason for this increased oxidation of protein, especially BCAA, is not clear, nevertheless, oxidation of BCAA may be significant because generates the amino acids alanine and glutamine, which can be transported from muscle to be used as fuels elsewhere. Alanine is carried to the liver where it contributes to the formation of glucose. Glutamine is a known fuel for the kidney and intestine. Whatever the reason, it appears that increased oxidation of protein and BCAA during exercise is obligatory.

One of the functions for the oxidation of BCAA in exercising muscle is, in effect, to remove lactate from muscle. It is well known that strenuously exercising muscle burns glucose in a largely anaerobic manner, resulting in the generation of lactate (lactate is derived directly from pyruvate). Build up of lactate in muscle is associated with muscle fatigue, and is considered to be undesirable.

The amino groups of the BCAA are transferred via intermediate reactions, to pyruvate, resulting in the formation of alanine. Alanine is exported to the liver to participate in glucose synthesis. Therefore, the pyruvate is involved in alanine synthesis is not converted to lactate. Therefore, BCAA oxidation serves to modulate lactate accumulation in muscle.

In addition, protons H⁺ from catabolic reactions must be eliminated, so as to remove any risk of a drop in pH. The proton is removed from muscle by combining (in the form of ammonium—NH⁺₄) with glutamate to form glutamine. When taken up by the kidney, NH⁺₄ (and hence H⁺) is removed and excreted in the urine.

Equally important, BCAA administration given before and during exhaustive aerobic exercise to individuals with reduced muscle glycogen stores may also delay muscle glycogen depletion. Although there are numerous reported metabolic causes of fatigue such as glycogen depletion, proton accumulation, decreases in phosphocreatine levels, hypoglycemia, and increased free tryptophan/BCAA ratio, it is the increase in the free tryptophan/BCAA ratio that may be attenuated with BCAA supplementation.

During prolonged aerobic exercise, the concentration of free tryptophan increases and the uptake of tryptophan into the brain increases. When this occurs, 5-hydroxytryptamine (a.k.a. serotonin), which is thought to play a role in the subjective feelings of fatigue, is produced. Similarly, BCAAs are transported into the brain by the same carrier system as tryptophan and thus “compete” with tryptophan to be transported into the brain. Therefore, it is believed that when certain amino acids such as BCAAs are present in the plasma in sufficient amounts, it theoretically may decrease the uptake of tryptophan in the brain and ultimately decrease the feelings of fatigue.

A further observation concerns the BCAA and the liver. It is known that during strenuous exercise the liver suffers a net loss of the BCAA because the skeletal muscle takes up BCAA from the blood. It has been noted that the rate of protein breakdown in the liver can be partly reversed by amino acids—in particular glutamine.

Glutamine is also known as 2-aminoglutaramic acid, levoglutamide, (S)-2, 5-diamino-5-oxopentaenoic acid and glutamic acid 5-amide and is included in this invention, to provide the liver with an amino acid to encourage protein synthesis, in the proper metabolic environment. The liver is central to numerous metabolic functions and is important in the adaptation to exercise.

Glutamine is the most abundant amino acid found in muscle and has important functions as a precursor for the synthesis of other amino acids. Many cells required for immune function rely on glutamine as source for energy production. Physical activity can deplete glutamine levels, and as such, glutamine is often considered to be a ‘conditionally essential’ amino acid. It has been shown that glutamine levels of groups involved in several different types of activities or sports found that powerlifters and swimmers had lower glutamine levels than cyclists and non-athletes, suggesting that high resistance load activities require increased amounts of glutamine. Moreover, this depletion of glutamine has been linked to immunosuppression often resulting from intense training. Supplementation with glutamine in conjunction with additional antioxidants can increase body weight, body cell mass, and intracellular water when compared with placebo. Glutamine is also capable of stimulating insulin secretion. Although the present invention is not to be bound by any theoretical explanation, it is believed that L-glutamine also aids in muscle recovery, improves endurance, reduces fatigue, and strengthens the immune system.

L-arginine is considered a semi-essential amino acid. Normally L-arginine is synthesized in sufficient amounts by the body. However, conditions and circumstances are known wherein additional L-arginine supplementation is required. As a precursor to nitric oxide, L-arginine plays an important role in regulating cardiovascular endothelium-dependent processes. Many of the therapeutic effects of L-arginine are likely due to its role as a NO precursor. Additionally, L-arginine has been shown to increase aerobic exercise capacity and NO production.

Nitric oxide is a free radical which is generated in biological systems. Nitric oxide synthase produces NO through the catalysis of a five-electron oxidation of the guanidine nitrogen of L-arginine. In this process, L-arginine is oxidized to L-citrulline via two successive monoxygenation reactions. In the first reaction, NOS acts with NADPH and O₂ to produce N^{ω} hydroxy L-Arginine as an intermediate in the overall reaction. N^{ω}hydroxy L-Arginine is then further oxidized to form L-citrulline and NO. The reaction takes places in the endothelial cells where Ca⁺⁺-calmodulin, NADPH, tetrahydrobiopterin, FAD and FMN are required as co-factors.

Endogenous NO functions include the signaling of vasodilation of blood vessels. Vasodilation leads to increased blood flow, particularly in working skeletal muscle, wherein an increase in the transport of nutrients and waste products is seen. Nutrient requirements and waste products increase with increasing muscle metabolism. Therefore, increased vasodilation will assist in the transport of skeletal muscle requirements and metabolic products during periods of exercise.

Creatine (also known as N-methyl-N-guanyl glycine or (alpha methyl guanido) acetic acid) is an amino acid compound produced naturally in the liver and kidneys from the amino acids glycine, arginine, and methionine and found in some foods such as meat and fish.

Creatine replenishes energy stores in working muscle cells. The resultant increase in muscular energy stores from creatine supplementation in an individual, combined with physical exercise leads to increased strength, and a reduction in fatigue resulting from high-intensity exercise as well as increasing muscle growth.

About 65% of creatine is stored in muscle as phosphocreatine (creatine bound to a phosphate molecule). Muscular contractions are fueled by the dephosphorylation of adenosine triphosphate (ATP) to produce adenosine diphosphate (ADP) and without a mechanism to replenish ATP stores, the supply of ATP would be totally consumed in 1-2 seconds. Phosphocreatine serves as a major source of phosphate from which ADP is regenerated to ATP. Six seconds following the commencement of exercise, muscular concentrations of phosphocreatine drop by almost 50% and creatine supplementation has been shown to increase the concentration of creatine in the muscle and further said supplementation enables an increase in the resynthesis of phosphocreatine leading to a rapid replenishment of ATP within the first two minutes following the commencement of exercise. Through this mechanism creatine improves strength and reduces fatigue.

Creatine also mediates remarkable neuroprotection in experimental models of amyotrophic lateral sclerosis, Huntington's disease, Parkinson's disease, and traumatic brain injury. Also, oral creatine administration to experimental animals has been shown to increase protection and inhibition of cell-death cascades in neural tissue during periods of stress, such as ischemia. The protective effect of creatine administration has been linked to improved energy balance. This increased energy balance, preservation of ATP levels, inhibits cytochrome c release from the mitochondria. Release of cytochrome c signals caspase-3 activation and cell death often results. Administration of creatine conserves energy balance in neural cells during periods of stress, thereby preserving neural tissue by inhibiting caspase-mediated cell death.

For example, creatine or a derivative thereof may be present in various embodiments of the present invention as for example a hydrate, salt or ester thereof. The creatine which is employed in the compositions of the present invention preferably comprises creatine monohydrate. Creatine salts comprise malate, maleate, fumarate, tartrate, nitrate, succinate, pyruvate, pyroglutamate, glutamate, citrate, or any other pharmaceutically acceptable salt as known in the art.

The carbohydrates or sugar in the present invention can be a mixture of one or more monosaccharides or disaccharides, and preferably in combination with one or more complex carbohydrates. In selecting effective carbohydrates and carbohydrate levels for use in the present compositions, it is important that the carbohydrates and levels thereof which are chosen allow a sufficient rate of digestion and intestinal absorption to provide a steady maintenance of glucose, which in turn provides energy and alertness to the consumer.

Monosaccharides and disaccharides provide immediate energy to the consumer while the complex carbohydrate components, are hydrolyzed in the digestive tract to provide a later, or delayed and maintained, onset of energy for the consumer. The monosaccharide utilized herein is a molecule of the general formula CₙH₂Oₙ, wherein n is an integer equal to or greater than 3. The monosaccharide herein is digestible, i.e., capable of metabolism by a mammalian body. Non-limiting examples of monosaccharides which may be utilized herein include sorbitol, mannitol, erythrose, threose, ribose, arabinose, xylose, xylitol, ribulose, glucose, galactose, mannose, fructose, and sorbose. Preferred monosaccharides for use herein include glucose and fructose, most preferably glucose.

One or more disaccharides may also be used as a source of immediate energy. The disaccharide utilized herein may be a molecule of the general formula CₙH₂ₙ₋₂Oₙ₋₁, wherein the disaccharide has 2 monosaccharide units connected via a glycosidic bond. In such formula, n is an integer equal to or greater than 3. Non-limiting examples of disaccharides which may be utilized herein include sucrose, maltose, lactitol, maltitol, maltulose, and lactose. The most preferred disaccharide for use herein is sucrose.

The invention may also include a complex carbohydrate such as an oligoosaccharide, polysaccharide, and/or carbohydrate derivative, preferably an oligosaccharide and/or polysaccharide. As used herein, the term “oligosaccharide” means a digestible linear molecule having from 3 to 9 monosaccharide units, wherein the units are covalently connected via glycosidic bonds. As used herein, the term “polysaccharide” means a digestible (i.e., capable of metabolism by the human body) macromolecule having greater than 9 monosaccharide units, wherein the units are covalently connected via glycosidic bonds. The polysaccharides may be linear chains or branched. Preferably, the polysaccharide has from 9 to about 20 monosaccharide units. Carbohydrate derivatives, such as a polyhydric alcohol (e.g., glycerol), may also be utilized as a complex carbohydrate herein. As used herein, the term “digestible” means capable of metabolism by enzymes produced by the human body.

Examples of preferred complex carbohydrates include raffinoses, stachyoses, maltotrioses, maltotetraoses, glycogens, amyloses, amylopectins, polydextroses, and maltodextrins. The most preferred complex carbohydrates are maltodextrins.

Maltodextrins are a form of complex carbohydrate molecule which is several glucose units in length. The maltodextrins are hydrolyzed into glucose in the digestive tract where they provide an extended source of glucose. Maltodextrins may be spray-dried carbohydrate ingredients made by controlled hydrolysis of corn starch.

Carbohydrate ingestion is known to stimulate the secretion of insulin which in turn facilitates the uptake of glucose into skeletal muscle via glucose transporter 4 (GLUT4) translocation. Glucose is then converted to and stored as glycogen and triglycerides. Moreover, insulin also plays an important role in protein metabolism where it inhibits the breakdown of protein or proteolysis. Furthermore, insulin promotes the uptake of amino acids into muscle and stimulates protein synthesis, particularly following exercise. Insulin has also been shown to stimulate creatine uptake by muscle cells. Creatine retention has been shown to be markedly improved by the concomitant ingestion with carbohydrates. Carbohydrate ingestion augments skeletal muscle creatine accumulation during creatine supplementation in humans. Thus, the ingestion of creatine combined with a source of carbohydrates is recommended to improve retention. Dextrose is a simple sugar or monosaccharide commonly known as D-glucose. Also known as ‘grape sugar’ or ‘blood sugar’, it is found mainly in honey and fruits and is a building-block of glycogen, cellulose and starch. Recently, dextrose was shown to boost the performance a female rowers as compared to ribose, which had been theorized to replenish muscle energy.

The inflammatory response is a predictable phenomenon that accompanies musculoskeletal injury. While this response is a necessary component of the healing process, uncontrolled inflammation may prolong skeletal muscle recovery after intense exercise or training induced injury. Consequently, this type of muscle injury may delay return to normal function.

Proteases are a group of biologically active enzymes responsible for initiating protein catabolism via hydrolysis of peptide bonds that link amino acids together in a polypeptide chain. There are four primary groups of proteases grouped based on function and catalytic site of action: serine proteases, cysteine proteases, aspartic acid proteases, and metalloproteases.

Members of these groups can act as either exopeptidases responsible for cleavage of terminal amino acids (e.g., aminopeptidases or carboxypeptidases) or as endopeptidases responsible for attacking internal peptide bonds (e.g., trypsin, chymotrypsin, pepsin, papain, etc.). These enzymes are involved in physiological processes ranging from the digestion of dietary protein to blood clotting or immunological function.

Dietary supplementation with oral proteases may attenuate losses in skeletal muscle force production as well as muscle soreness after strenuous exercise. Reduction in muscle damage, delayed onset of muscle soreness, and/or force decrements using protease is achieved via regulation of inflammatory process after exercise.

It is believed that proteases regulate the postexercise inflammatory process by a reduction in the biosynthesis of prostaglandins and other eicosanoids via inhibition of the arachidonic acid cascade and a reduction in swelling and edema by improved mobilization of inflammatory cells from the tissues. After an injury or strenuous exercise, some of these capillaries may be damaged, making them incapable of carrying fluid to and from the affected tissue. The tissue damage increases cell permeability and allows leakage of proteins such as fibrinogen, albumin and various globulins into the interstitial spaces. The edema is thought to activate type IV sensory neurons resulting in the sensation of dull, diffuse pain commonly associated with delayed onset of muscle soreness.

Proteolytic enzymes reduce the amount of fibrin in the damaged capillary, improve circulation and speed healing. Once in the blood stream proteolytic enzymes hydrolyze (digest) the fibrin network and enhance blood flow. Additionally, these same proteases have been known to stimulate phagocytes (cells that ingest foreign particles and debris) and accelerate elimination by way of the lymphatic system. Once fibrin is removed, the cells can rapidly recover.

Furthermore oral protease ingestion is expected to attenuate muscle damage and improve skeletal muscle function by reducing circulating macrophages and neutrophils after strenuous exercise and by regulating the effects of pro-inflammatory mediators such as tumor necrosis factor alpha (TNF-α) and interleukin 1A (IL-1 α). In summary, the anti-inflammatory action of protease supplementation increases tissue permeability, facilitates resorption of edema, hydrolyzes and removes extra-cellular proteins damaged by free radicals (which are especially susceptible to proteolysis), and accelerates repair of damaged muscle tissue.

Papain is a protein-cleaving enzyme derived from papaya (Carica papaya) among other plants. Papain and chymopapain can be found in the latex of the papaya plant and its green fruits. In addition to papain's proteolytic functions, it also has a mild, soothing effect on the stomach. It is most often used as a meat tenderizer and is the most widely studied of the cysteine proteases because of this commercial value.

Bromelain is a glycoprotein enzyme with proteolytic activity derived from the stem of the pineapple plant (Ananas comosos). Bromelain has anti-inflammatory properties and has been successful at improving the symptoms of arthritis and knee pain. Bromelain has been theorized to be due multiple activities unrelated to its proteolytic activity, including the ability to block various chemokine mediators of inflammation.

The proteases may be obtained from animal, plant or microbial sources. For example, Proteases 4.5 and 6.0 from Aspergillus oryzae have both exo-peptidase and endo-peptidase activity and are unusually stable and active throughout a wide range of pH conditions (i.e., a pH of 2.0-10.0). Protease 4.5 works with endogenous enzymes to provide protein digestion in the stomach and pyloric regions of the small intestine and Protease 6.0 works with endogenous enzymes to provide protein digestion through all portions of the digestive tract.

Amylase is an enzyme that changes complex sugars (starches) into simple sugars during digestion. Amylase is secreted by the salivary glands and the pancreas that helps in the digestion of carbohydrates

White willow bark (Salix alba) is a source of salicin, a precursor of acetylsalicylic acid (aspirin) traditionally used to treat pain, fever and inflammation. White Willow bark extract has been shown to be as effective as a synthetic NSAID for relieving lower back pain. As a source of salicin, the anti-inflammatory mechanism of White Willow bark is understood to be mediated via the inhibition of cyclooxygenase. It is herein understood that White Willow bark will reduce pain and inflammation by blocking the activity of cyclooxygenase enzymes and inhibit the production of mediators of inflammation.

The composition may further comprise an anti-emetic to suppress the general feeling of nausea that can often arise after a strenuous physical workout. The exercise-induced nausea may also suppress the desire to ingest food after exercise which is counterproductive to an athlete desirous of gaining muscle mass because this situation would result in catabolism over anabolism.

Examples of anti-emetics include, but are not limited to ginger, basil, peppermint, Atractylodes, Berberis, cloves, fennel, oregon grape, red raspberry, wild yam and elecampane (Inula helenium).

Ginger (Zingiber officinale) is effective for treating nausea caused by seasickness and morning sickness. Ginger additionally helps to calm the gut and protect the gastric system by reducing stomach acidity through the increase of the pH of stomach acid. As a result, it reduces the rate of gastric secretions and boosts digestive enzyme activity. Ginger also reduces bloating and constipation. In fact, studies have shown that ginger regulates peristalsis, thus it helps to tone and strengthen the muscles involved in digestion, promoting overall health.

The composition may further comprise a calming agent to promote feeling of relaxation after strenuous exercise. Often times after a workout, athletes have difficulty resting and such agents will provide a calming sensation to encourage rest. Non-limiting calming agents include theanine, Rhodiola Rosea, russian tarragon, Withania Somnifera, Humulus lupulus, Eclipta alba, Nardostachys jatamansi, Lavandula officinalis or Passiflora coerulea.

Theanine (gamma-glutamylethylamide) is an amino acid found in green tea. It is however distinct from the polyphenols and catechins that are typically associated with the beneficial effects of green tea. While catechins are generally associated with antioxidant activity, theanine is associated with anti-stress and cortisol control. In hypertensive rats, theanine has been shown to lower blood pressure. Furthermore, weight gain and fat accumulation have been suppressed in rats fed theanine relative to control animals.

Rhodiola Rosea is also known as ‘Golden root’, ‘Arctic root’ and Crenulin. Rhodiola Rosea confers increased resistance to multiple stresses, both mental and physical. The mechanism of action for Rhodiola Rosea appears to be primarily its ability to increase the levels of monamine neurotransmitters such as serotonin, dopamine and norepinephrine. In human clinical trials, Rhodiola Rosea has been shown to improve mental performance and reduce stress-induce fatigue without side-effects. Furthermore, Rhodiola Rosea may also improve endurance exercise performance.

Russian tarragon (Artemisia dracunculus) is a perennial herb widely used in cooking. Historically it has been used a treatment for headaches and dizziness. Additionally, an essential oil extracted from Artemisia dracunculus may have potential therapeutic effects as an anticonvulsant and mild sedative.

Withania somnifera (Ashwagandha, Winter Cherry) is an herb used in traditional East Indian medicine. Withania somnifera is reported to have a number of beneficial effects including antioxidant and anti-stress. In rats, Withania Somnifera has a positive effect on mood by reducing stress and anxiety. In addition, Withania somnifera has been shown to attenuate both age-associated and chemical-induced cellular and tissue oxidative damage in rats and may be useful in the treatment of osetoarthritis.

The Hop plant (Humulus lupulus) is a flowering vine used traditionally as a sedative for anxiety and sleep difficulties. In mice, Hops extract has been shown to have sleep-enhancing and antidepressant activities. Additionally, in rats, administration of Hops extract has been shown to produce sedative effects. Hops extract has been shown to modulate the gamma-aminobutyric acid receptor (GABA(A) receptors) and display GABA-like activity. GABA is an inhibitory neurotransmitter that can induce relaxation and sleep. Modulation of any or all of these receptors may mediate the sleep-inducing activity of Hops.

The Eclipta alba plant grows as a weed in Asia and South America. It has been used in traditional medicine for many treatments. Eclipta alba extract has been shown to possess nootropic effects and attenuate stress-induced neurochemical changes in animal studies as well as have analgesic effects.

Nardostachys jatamansi herb is part of traditional Indian medicine where it is used for treatments affecting the central nervous system, particularly for depressant action. The effect of acute and subchronic administration of an extract of the roots of Nardostachys jatamansi has been shown to significantly increase serotonin (5-HT) in Wistar rats. Serotonin is a neurochemical, concentrated in the raphe nucleus region of the brain wherein its axon project into several areas of the brain such as the hypothalamus. Through its resultant actions on the hypothalamus, serotonin is understood to be involved in inducing sleep and control of mood, among other function. In fact, many well-known antidepressants act by inducing serotonin release or inhibiting its re-uptake in the brains. It is herein understood by the inventors that increasing the amount of serotonin promotes a feeling of calmness in an individual.

Oil from Lavandula officinalis, commonly known as the Lavender plant is frequently used in aromatherapy as a mode to induce relaxation. The mild sedative effects of Lavender have been demonstrated in animals and humans.

Passion flower (Passiflora coerulea) has been used traditionally for relaxation and as a sleep-aid as well as a treatment for anxiety. The main active component of passion flower is thought to by chrysin, one of several flavonoids which have been isolated from this plant. In mice, chrysin has been shown to act as an agonist of benzodiazepine receptors and also possess anti-anxiety activity

In aspects, the present invention also relates to compositions and methods that sustain and promote metabolic energy levels. More particularly, the present invention relates to nutrient compositions and methods that sustain and promote positive metabolic energy levels in a targeted manner.

Embodiments provide compositions and methods that sustain and promote metabolic energy levels through targeting specific body tissues. That is, they attempt to provide energy to skeletal muscle so as to make possible an increase in the rate and intensity of muscle use. Because vigorous muscle exercise causes damage to the muscle tissue and its cells, the body repairs the cells after damage. When the muscle cells are repaired, they grow: their size may be increased (e.g., protein synthesis) and the number of cellular components per cell may be increased in order to resist future damage. Further exercise, if effective, must in a sense damage the repaired cells in order to further promote growth and subsequent repair.

Since each stage of this exercise-damage-repair growth cycle requires energy, the greater the muscle development, the greater the energy requirements for the cycle. Thus, in aspects, the nutritional supplements of the present invention provide useful energy to the muscle for the cycle. For example, if energy can be supplied so as to allow for increased exercise, then increased damage will result and so growth (growth as used herein is intended to include: hypertrophy or physical size of a muscle fiber, as occurs with Type Ia and IIb (also known as fast twitch oxidative and fast twitch glycolytic) muscle fibers; intracellular density, such as occurs with Type I fibers (also known as slow twitch oxidative); as well as hyperplasia, which appears to occur with various types of fibers through various mechanism, e.g. fiber splitting, satellite cell adaptation, stem cell adaptation, etc.).

Ginseng has been revered in China as the King of Herbs for centuries. It helps to replenish the qi, or life force, of the body through a number of mechanisms. Ginseng is an adaptogen, which means that it works with the body to help restore balance. Chinese practitioners use it as a tonic to increase physical strength and energy and promote the proper functioning of the body's organs. Ginseng also treats fatigue and builds stamina and endurance by enhancing the body's ability to adapt to stress.

Panax ginseng is preferred, however, other forms of ginseng may alternatively be used and such other forms include, but are not limited to, Panax quinquefolius, Panax notoginseng, Eleutherococcus senticosus, and Acanthopanax senticosus. It strengthens the heart muscle and stimulates the immune system. It also increases cerebral circulation, which enhances memory, alertness and other cognitive functions. Some of the active principles in the herb, known as ginsenosides, have antioxidant properties as well. The preferred composition contains lesser amounts of Panax quinquefolius, also known as American ginseng. This herb has stress-reducing qualities that help to balance the warmer properties of Panax ginseng. It also promotes proper functioning of the immune system.

Vitamins are vital elements to obtain a proper metabolism. The vitamin B family, for example, helps the body and mind in many ways. For example, vitamin B12 and vitamin B6 regenerate red blood cells.

Vitamin B1 improves mental attitude, and keeps the nervous system, muscles, and heart functioning normally.

Vitamin B2 plays a role in cosmetic care by promoting healthy skin, nails and hair. Niacin is a member of the B-complex family. Niacin promotes a healthy digestive system, reduces high blood pressure, and increases energy through proper utilization of food.

Vitamin B6 is involved in a wide variety of metabolic functions in the body. For example, Vitamin B6 is necessary for synthesis of more than 100 enzymes that assist in protein metabolism. Vitamin B6 has been shown to amplify the functions of peptides and amino acids in the body, demonstrating synergistic reactions with a number of amino acids. Vitamin B6 is also necessary for the manufacture of hemoglobin—that portion of blood responsible for the transport of oxygen throughout the body. Vitamin B6 has also been shown to actually increase the oxygen carrying capacity of hemoglobin, a very important factor in improving athletic performance. Vitamin B6 has also been shown to increase cell growth, including muscle tissue cells. The increased cell growth translates directly to faster muscle tissue repair times and faster recovery times after exercise.

Vitamin B6 has been shown to assist in the conversion of stored carbohydrates to glucose for use in manufacturing energy required by a hard training body. Vitamin B6 is also a necessary agent for the body to manufacture carnitine, a nitrogen containing, short chain carboxylic acid. The most common form of Vitamin B6, pyridoxine hydrochloride, is preferred.

Studies have demonstrated that carnitine assists in the transport of fats from storage to the mitochondria where the fat is metabolized for energy. In sports nutrition; it is thought that the enhanced fat oxidation promoted by carnitine spares stored carbohydrates, allowing them to be utilized at more convenient times such as during hard training. Recently, it has been shown that carnitine may reduce lactic acid production in muscle tissue during aerobic exercise, thus leading to decreased recovery times after exercise.

Antioxidants are compounds that decrease protein oxidation (e.g. prevent formation of protein carbonyls). They may be sources of thiols (e.g. Lipoic acid, cysteine, cystine, methionine, S-adenosyl-methionine, taurine, glutathione and natural sources thereof), or compounds that upregulate their biosynthesis in vivo, for example.

The antioxidants according to the invention may be used either alone or in association with other antioxidants such as vitamin C, vitamin E (tocopherols and tocotrienols), carotenoids (carotenes, lycopene, lutein and zeaxanthine) ubiquinones (e.g.CoQ10), tea catechins (e.g. epigallocatechin gallate), coffee extracts containing polyphenols and/or diterpenes (e.g. kawheol and cafestol), ginkgo biloba extracts, grape or grape seed extracts rich in proanthocyanidins, spice extracts (e.g. rosemary), soy extracts containing isoflavones and related phytoestrogens and other sources of flavonoids with antioxidant activity, compounds that upregulate cell antioxidant defense (e.g. ursodeoxycholic acid for increased glutathione S-transferase, ursolic acid for increased catalase, ginseng and ginsenosides for increase superoxide dismutase).

There is also a need for a nutritional supplement that increases muscle strength after exercise during periods when training efforts have to be stopped because of injuries or holidays. It is frequently observed that muscle mass that was built up during exercise rapidly decreases, and a lot of time is normally required to regain the level that was previously present. Losses in lean body mass are also observed frequently in persons that have to be inactive for quite a while, e.g. because they have to stay in bed to injury, disease or other disorders. Thus, there is also a need for nutritional supplements that help prevent losses of body mass during these periods.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is a powdered beverage mix.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

According to various embodiments of the present invention, the botanical ingredients may be the whole plant or parts of the plant or extracts from the whole plant or parts of the plant. The extraction process may be carried out using methods known in the art, including but not limited to solvent extraction, percolation, vat extraction, or countercurrent extraction. The degree of comminutation of the plant material prior to the extraction process should provide sufficient particulate surface for the extraction solvent to contact the material.

Extraction may be at ambient temperature or at elevated temperature. The resulting extract solution is then dried to substantially remove the solvent.

The inclusion of specific excipients, as well as specific dosage formats may be utilized to achieve specific controlled-release of active ingredients. Such formats include but are not limited to quick-release, timed-release, slow-release and delayed-release.

The present nutritional composition or those similarly envisioned by one of skill in the art, may be utilized in methods to improve skeletal muscle protein synthesis. As such, the present invention may be utilized as a sole means of improving skeletal muscle protein synthesis or in combination with other like-directed compounds.

### EXAMPLE 1

In this example, an athlete consumes one serving of the food supplement as described before sleep. This regime is continued for four days in order to enhance muscle size and/or strength. Each serving of the food supplement is approximately 42 grams.

Each approximate 42 gram serving is mixed with 12 ounces of cold water to provide a liquid drink. An additional 8 ounces of water is consumed after the food supplement liquid drink is consumed.

### EXAMPLE 2

In this example, an athlete consumes one serving of the food supplement as between exercise. This regime is continued for four days in order to enhance muscle size and/or strength. Each serving of the food supplement is approximately 42 grams.

Each approximate 42 gram serving is mixed with 12 ounces of cold water to provide a liquid drink. An additional 8 ounces of water is consumed after the food supplement liquid drink is consumed.

In the foregoing specification, the invention has been described with specific embodiments thereof; however, it will be evident that various modifications and changes may be made thereto without departing from the broader spirit and scope of the invention.

We claim:

1. A comestible composition comprising casein, creatine or derivatives thereof, at least one branched chain amino acid and at least one protease.

2. The composition according to claim 1 wherein the at least one branched chain amino acid is selected from the group consisting of L-leucine, L-isoleucine, L-valine and combinations thereof.

3. The composition according to claim 2 wherein the at least one protease is an endopeptidase.

4. The composition according to claim 3 wherein the endopeptidase is trypsin, chymotrypsin, pepsin, papain or bromelain.

5. The composition according to claim 4 wherein the endopeptidase is papain.

6. The composition according to claim 4 further comprising carbohydrate.

7. The composition according to claim 6 wherein the carbohydrate is dextrose.

8. The composition according to claim 6 wherein the derivatives of creatine is a hydrate, salt or ester.

9. The composition according to claim 8 wherein the creatine salt is malate, maleate, fumarate, tartrate, nitrate, succinate, pyruvate, pyroglutamate, glutamate or citrate.

10. The composition according to claim 6 further comprising L-glutamine.

11. The composition according to claim 10 further comprising L-arginine.

12. The composition according to claim 11 further comprising amylase.

13. The composition according to claim 12 further comprising at least one anti-emetic.

14. The composition according to claim 13 wherein the anti-emetic is ginger, basil, peppermint, Atractylodes, Berberis, cloves, fennel, oregon grape, red raspberry, wild yam or elecampane (Inula helenium).

15. The composition according to claim 13 further comprising at least one calming agent.

16. The composition according to claim 15 further wherein the calming agent is theanine, Rhodiola Rosea, Artemisia dracunculus, Withania somnifera, Humulus lupulus, Eclipta alba, Nardostachys jatamansi, Lavandula officinalis or Passiflora coerulea.

17. The composition according to claim 15 further comprising at least one adaptogen.

18. The composition according to claim 17 wherein the adaptogen is Panax ginseng, Panax quinquefolius, Panax notoginseng, Eleutherococcus senticosus or Acanthopanax senticosus.

19. The composition according to claim 17 further comprising at least one vitamin.

20. The composition according to claim 19 wherein the vitamin is vitamin B6.

21. A method for increasing muscle mass after physical exercise comprising providing a comestible composition comprising casein, creatine or derivatives thereof, at least one branched chain amino acid and at least one protease before sleep for four days.
